(19) United States
(12) Patent Application Publication
(10) Pub. No.: US 2013/0267854 A1
(43) Pub. Date: Oct. 10, 2013

(54) Optical Monitoring and Computing Devices and Methods of Use

(71) Applicants: Jami Johnson, Boise, ID (US); Michelle Sabick, Boise, ID (US)

(72) Inventors: Jami Johnson, Boise, ID (US); Michelle Sabick, Boise, ID (US)

(21) Appl. No.: 13/442,551

(22) Filed: Apr. 9, 2012

(51) Int. Cl. A61B 5/00

(52) U.S. Cl. CPC A61B 5/0082
USPC 600/473; 600/479

(57) ABSTRACT

The present invention relates to medical devices and, in particular, to optical computing devices configured to monitor cardiac-related conditions. One optical computing device includes a substrate, at least one light source mounted on the substrate and configured to emit electromagnetic radiation that optically interacts with a vasculature and generates an optically interacted signal, a plurality of detectors mounted on the substrate and configured to detect the optically interacted signal, and a stabilizing matrix arranged on the substrate and substantially surrounding the at least one light source and the plurality of detectors. The stabilizing matrix may be configured to absorb vibration and thereby reduce motion artifacts detectable by the plurality of detectors.

### BACKGROUND

The present invention relates to medical devices and, in particular, to optical computing devices configured to monitor cardiac-related conditions.

Photoplethysmography (PPG) is a noninvasive and low cost optical technique used for studying skin blood volume pulsations. Blood-pressure waves that are generated by the heart propagate along the skin arteries, locally increasing and decreasing the tissue blood volume with the periodicity of heartbeats. PPG exploits this phenomenon through the use of narrow-band light-emitting diodes (LEDs) in the infrared or near-infrared region. Back scattering of the optical radiation is typically detected in either transmission or reflection configuration by one or more strategically-placed photodetectors. Heart rate, respiratory rate, and tissue blood perfusion, as well as indicators of cardiac disorders and peripheral vascular diseases can be extracted from the analysis of a single PPG trace. Factors such as skin color, volume of adipose tissue, ambient light, sensor location, and movement artifacts have been known to affect the robustness and consistency of PPG signals.

Of late, there has been a resurgence of interest in using PPG, driven primarily by the demand for low cost, simple, and portable technology for the primary care and community-based clinical settings, and the wide availability of inexpensive and small semiconductor components. PPG technology has been used in a wide range of commercially available medical devices for measuring oxygen saturation, blood pressure and cardiac output, assessing autonomic function, and also detecting peripheral vascular disease. As a result, innovative methods or devices capable of obtaining reliable PPG signals in various locations on the body have the potential to be useful in various clinical applications, as well as for self-monitoring applications.

### SUMMARY OF THE INVENTION

The present invention relates to medical devices and, in particular, to optical computing devices configured to monitor cardiac-related conditions.

In some aspects of the disclosure, a device is disclosed. The device may include a substrate and at least one light source mounted on the substrate and configured to emit electromagnetic radiation that optically interacts with a vasculature and generates an optically interacted signal. The device may also include a plurality of detectors mounted on the substrate and configured to detect the optically interacted signal, and a stabilizing matrix arranged on the substrate and substantially surrounding the at least one light source and the plurality of detectors. The stabilizing matrix may be configured to absorb vibration and thereby reduce motion artifacts detectable by the plurality of detectors.

In some aspects, another device may be disclosed. The device may include a housing having a front surface and a back surface, and a substrate having a front side and a back side, where the back side may be removably coupled to the back surface of the housing. The device may also include at least one light source mounted on the front side of the substrate and configured to emit electromagnetic radiation that optically interacts with a vasculature and thereby generates an optically interacted signal. A plurality of detectors may be mounted on the front side of the substrate and configured to detect the optically interacted signal. The device may further include a stabilizing matrix arranged on the front side of the substrate and substantially surrounding the at least one light source and the plurality of detectors. The stabilizing matrix may be configured to absorb vibration and thereby reduce motion artifacts detectable by the plurality of detectors.

In some aspects of the disclosure, a method for detecting cardiac-related conditions is disclosed. The method may include emitting electromagnetic radiation through a vasculature using at least one light source mounted on a substrate. The electromagnetic radiation may be configured to optically react with the vasculature and reflect an optically interacted signal. The method may also include detecting the optically interacted signal with a plurality of detectors mounted on the substrate. The plurality of detectors may be configured to generate signal data. The method may further include absorbing vibration and reducing motion artifacts detectable by the plurality of detectors with a stabilizing matrix, where the stabilizing matrix may be arranged on the substrate and substantially surrounding the at least one light source and the plurality of detectors. The method may even further include receiving the signal data with a processing device communicably coupled to the plurality of detectors, and processing the signal data to determine the cardiac-related conditions.

The features and advantages of the present invention will be readily apparent to those skilled in the art upon a reading of the description of the preferred embodiments that follows.

### BRIEF DESCRIPTION OF THE DRAWINGS

The following figures are included to illustrate certain aspects of the present invention, and should not be viewed as exclusive embodiments. The subject matter disclosed is capable of considerable modifications, alterations, combinations, and equivalents in form and function, as will occur to those skilled in the art and having the benefit of this disclosure.

FIG. 1a is an exemplary optical computing device, according to one or more embodiments disclosed.

FIG. 1b is a variation of the exemplary optical computing device of FIG. 1a, according to one or more embodiments disclosed.

FIGS. 2a, 2b, and 2c are side views of the exemplary optical computing device of FIG. 1, according to one or more embodiments disclosed.

FIG. 3a is an isometric view of an exemplary housing that may be used to receive and seat an optical computing device, according to one or more embodiments disclosed.

FIG. 3b illustrates the housing of FIG. 3a with an optical computing device arranged therein, according to one or more embodiments disclosed.

FIG. 3c illustrates the optical computing device of FIG. 3b having a stabilizing matrix applied thereto, according to one or more embodiments disclosed.

FIG. 4 illustrates an exemplary interface configured to provide real-time cardiac-related information, according to one or more embodiments.

### DETAILED DESCRIPTION

The present invention relates to medical devices and, in particular, to optical computing devices configured to monitor cardiac-related conditions. The various embodiments disclosed herein may be used in a variety of applications and in a variety of ways in order to detect, monitor, and/or report a range of cardiac-related conditions. For example, the disclosed optical computing devices may be useful for, but not limited to, determining oxygen concentration in blood vessels, determining an individual's blood pressure and/or heart rate, determining the general condition of the adjacent vasculature of an individual, determining calorie expenditure based on respiration, determining the general condition of heart valves in an individual, and determining an individual's base metabolic rate. The resulting output signal may be depicted in the form of a photoplethysmograph (PPG) trace that can be analyzed. Those skilled in the art will readily recognize additional useful applications for the optical computing devices, and several advantages that may be derived from the novel components and configurations discussed herein.

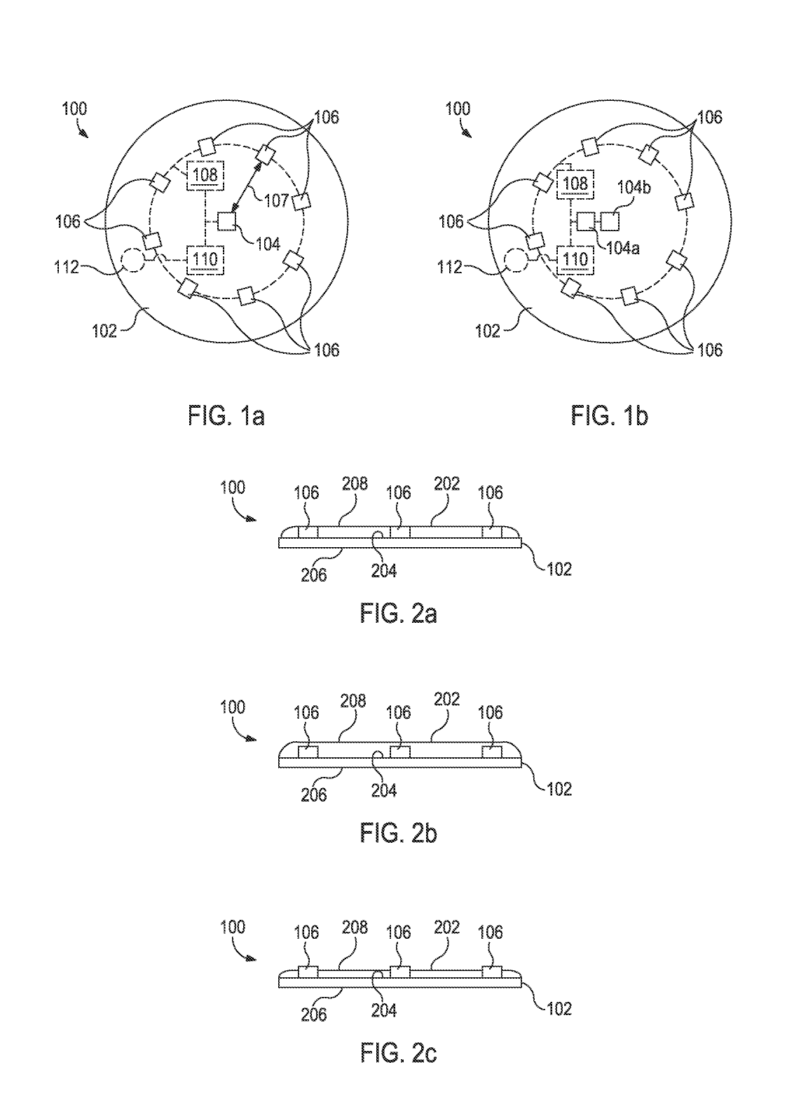

Referring now to FIG. 1a, illustrated is an exemplary optical computing device 100, according to one or more embodiments. In at least one embodiment, the device 100 may be generally characterized as a pulse oximeter, a photoplethysmograph, or similarly configured optoanalytical device. The optical computing device 100 may include a generally planar substrate 102 and at least one light source 104 mounted thereon or otherwise coupled thereto. The substrate 102 may be made of any rigid or semi-rigid material used to mechanically support and electrically connect the various components of the device 100. For example, the substrate 102 may be made of, but is not limited to, polymers, plastics, elastomers, metals, ceramics, combinations thereof, or the like. In some embodiments, the substrate 102 may be a printed circuit board. In other embodiments, however, the substrate 102 may be made of a flexible material so as to be able to generally conform to the contours or shape of the location on the body where the optical computing device 100 is utilized. Moreover, it should be noted that while FIG. 1a illustrates the substrate 102 in a generally circular shape, other shapes are also contemplated herein. For example, the substrate 102 may exhibit an oval, elliptical, or any suitable polygonal shape. In at least one embodiment, the substrate 102 may be octagonal.

The light source 104 may be any device capable of emitting or generating electromagnetic radiation. As used herein, the term “electromagnetic radiation” refers to visible light, ultraviolet light, red, infrared and near-infrared radiation, radio waves, microwave radiation, X-ray radiation and gamma ray radiation. In some embodiments, the light source 104 may be a light bulb, a light emitting device (LED), a laser, a photonic crystal, an X-Ray source, or the like. In at least one embodiment, the light source 104 may be an LED configured to emit red light (i.e., light having a wavelength between about 620 nm and about 740 nm) and/or infrared light (i.e., light having a wavelength between about 750 nm and about 1 mm). After being emitted from the light source 104, the electromagnetic radiation optically interacts with, for example, the vasculature of the individual and reflects an optically interacted signal. As used herein, “vasculature” means the circulatory system for passing nutrients, gases, hormones, blood cells, etc. to and from cells in order to maintain bodily homeostasis.

As illustrated, the light source 104 may be centrally-located on the substrate 102. In other embodiments, however, the light source 104 may be arranged at other locations on the surface of the substrate 102, without departing from the scope of the disclosure. As shown in FIG. 1b, the device 100 may equally include more than one light source 104, illustrated therein as light sources 104a and 104b. In one embodiment, the light sources 104a,b may be centrally-located on the substrate 102, as depicted. In other embodiments, however, the light sources 104a,b may be arranged in other relative configurations, without departing from the scope of the disclosure. Two or more light sources 104a,b may allow for the comparison of electromagnetic radiation absorption at different wavelengths. For example, the first light source 104a may emit infrared light and the second light source 104b may emit red light in order to measure the difference in absorption of oxy- and deoxygenated hemoglobin, respectively, which can be used to calculate oxygen saturation. In one embodiment, the light sources 104a,b may be configured to alternatingly turn on/off (i.e., pulsed) such that the detectors 106 are able to detect and measure the respective absorption rates. In other embodiments, however, one or more of the detectors 106 may be configured to detect wavelengths of infrared light while other detectors 106 are configured to detect wavelengths of red light in order to distinguish between the two signals. Potential other parameters that may be measured with two light sources 104a,b or varied detectors 106 include lipid plaque, fat, collagen, water, glucose, and elastin. Also, some colors may obtain a better signal depending on the individual. As can be appreciated, such alternative embodiments may be optimized for many physiological variables.

The device 100 may further include one or more detectors 106 mounted on the substrate 102 or otherwise coupled thereto. The detectors 106 may be optical detectors configured to detect the optically interacted signal reflected from the vasculature of an individual. Suitable detectors 106 for the device 100 may include, but are not limited to, phototransistors, photodiodes, photoresistors, photomultiplier tubes, photovoltaic cells, optical nanosensors, combinations thereof, or the like.

As illustrated, the detectors 106 may be configured to form a circular array about the light source 104. Since reflected light tends to scatter in a circular pattern, the circular array of detectors 106 may prove advantageous in enlarging the detection area of the device 100 and therefore increasing the probability of detecting a reflected signal. In other embodiments, however, the detectors 106 may be arranged in any other geometric configuration or arrangement so long as the light source 104 generally remains centrally-located with respect to the detectors 106. For example, the detectors 106 may equally be arranged in various polygonal configurations (e.g., square, rectangular, octagonal, etc.) and likewise provide adequate detection. Moreover, while FIGS. 1a and 1b depict the detectors 106 as being equidistantly offset from each other circumferentially, embodiments are contemplated herein where the detectors 106 are randomly offset from each other or otherwise arranged in a predetermined, non-equidistant fashion.

The detectors 106 may be radially-offset from the light source 104 by a predetermined distance 107. As can be appreciated, however, the predetermined distance 107 may be altered in varying embodiments of the device 100 in order to achieve desired reflectance parameters between the light source 104 and the detectors 106. For example, optical scattering may vary among individuals due to skin color, volume of adipose tissue, age, thickness of skin, location of the device 100 on the body, etc. Consequently, the predetermined distance 107 may be optimized in order to detect the best signal for a particular individual. Moreover, whereas a total of eight detectors 106 are illustrated in FIGS. 1a and 1b in the array, embodiments contemplated herein may include more or less than eight detectors 106 in an array, without departing from the scope of the disclosure. Moreover, in at least one embodiment, a second array (not shown) of detectors 106 may be included in the device 100 and be radially-offset from the first array of detectors 106. The second array may be used to supplement the first array of detectors 106 and thereby provide a more accurate resulting detection.

The device 100 may further include a power supply 108 and a processing device 110 (shown in phantom). In one embodiment, the power supply 108 and processing device 110 may each be arranged or otherwise mounted on the opposing side of the substrate 102 so as to not interfere with the communication between the light source 104 and detectors 106. The power supply 108 may be configured to provide power to the light source 104, the detectors 106, and the processing device 110 in order to properly operate the device 100. In one embodiment, the power supply 108 may include one or more rechargeable batteries, or the like. In other embodiments, however, the power supply 108 may be configured as an energy-scavenging device powered by kinetic energy derived from movement of the individual wearing the device 100. For example, movement of the individual may cause a magnet in an electromagnetic generator to move and thereby generate a rate of change of flux which results in some induced emf on adjacent coils, thereby generating a power output. The processing device 110 may be communicably coupled to each of the detectors 106 and configured to process the signal data received therefrom and thereafter provide an output for consideration by the user.

In some embodiments, the processing device 110 may be, for example, a general purpose microprocessor, a microcontroller, a digital signal processor, an application specific integrated circuit, a field programmable gate array, a programmable logic device, a controller, a state machine, a gated logic, discrete hardware components, an artificial neural network, or any like suitable entity that can perform calculations or other manipulations of data. In some embodiments, computer hardware can include elements such as, for example, a memory (e.g., random access memory (RAM), flash memory, read only memory (ROM), programmable read only memory (PROM), erasable read only memory (EPROM)), or any other like suitable storage device or medium. Executable sequences can be implemented with one or more sequences of code contained in the memory. In some embodiments, such code can be read into the memory from another machine-readable medium, such as a computer communicably coupled (either wired or wirelessly) to the processing device 110. As used herein, a machine-readable medium will refer to any medium that directly or indirectly provides instructions to a processor for execution.

Execution of the sequences of instructions contained in the memory can cause the processing device 110 to perform the process steps described herein. In addition, hard-wired circuitry can be used in place of or in combination with software instructions to implement various embodiments described herein. Thus, the present embodiments are not limited to any specific combination of hardware and/or software.

In other embodiments, the processing device 110 may instead be a wireless transmitter communicably coupled to the detectors 106 and configured to wirelessly communicate (e.g., via BLUETOOTH® technology or the like) the signal data received from the detectors 106 to an adjacent computing device adapted to filter and analyze the signal data and display any resulting cardiac-related data (e.g., a PPG trace). The adjacent computing device, such as a computer, personal digital assistant (PDA), smartphone, or the like, may be configured to receive and process the data received from the wireless transmitter and provide an output for consideration by the user. The smartphone, for example, may include an “app” which could be configured to process the received signals automatically. In other embodiments, however, one or more ports 112 may be defined on or otherwise provided by the device 100 in order to allow a wired connection directly to the adjacent computing device. In at least one embodiment, the ports 112 may further be utilized to provide power to the device 100, such as for recharging the power supply 108.

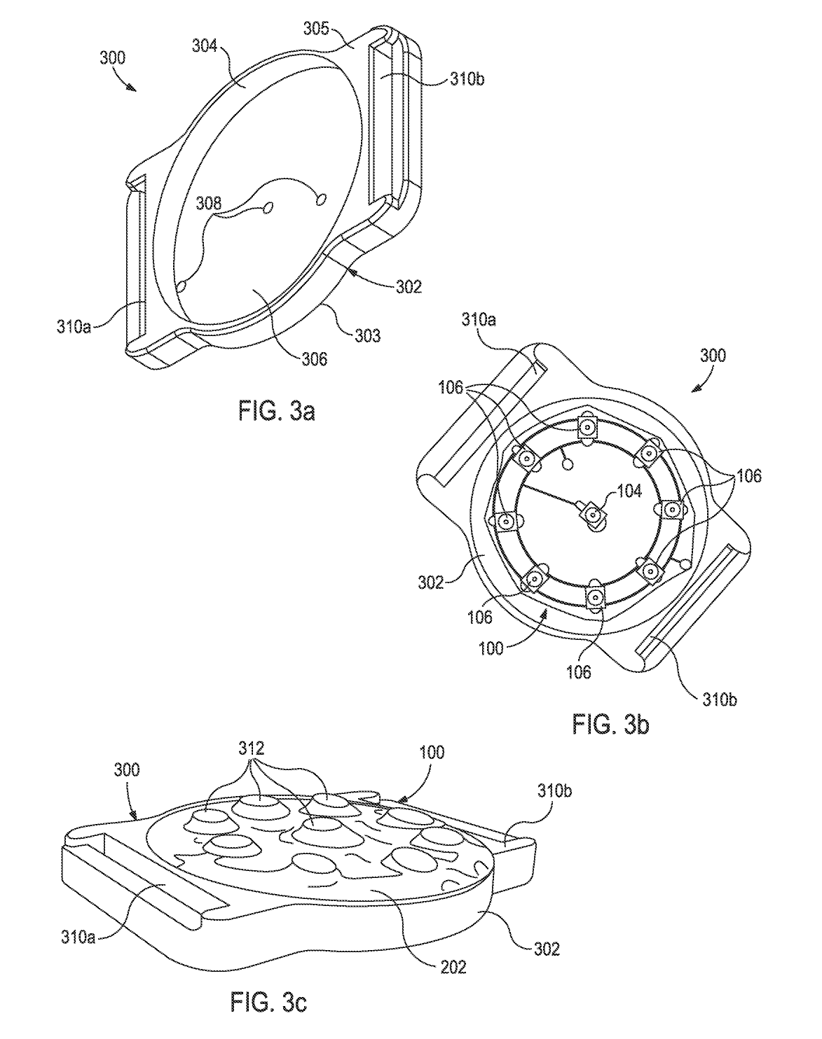

Referring now to FIGS. 2a, 2b, and 2c, with continued reference to FIGS. 1a and 1b, illustrated are a series of side views of the optical computing device 100. As depicted, the device 100 may include a front side 204 and a back side 206, where the detectors 106 and other electrical components are arranged on the front side 204. The device 100 may further include a stabilizing matrix 202 applied to or otherwise arranged on the front side 204 of the substrate 102 and thereby provide an outer surface 208. In some embodiments, the stabilizing matrix 202 may substantially surround or otherwise cover the various electrical components of the device 100, such as the detectors 106 and the light source 104 (not shown). In other embodiments, however, the stabilizing matrix 202 may be configured to surround the components, but the detectors 106 and/or the light source 104 may protrude a short distance past the outer surface 208 of the stabilizing matrix 202. For example, in FIG. 2a, the outer surface 208 of the stabilizing matrix 202 is illustrated as being flush with the detectors 106; in FIG. 2b, the stabilizing matrix 202 is illustrated as substantially covering the detectors 106; and in FIG. 2c, the detectors 106 protrude a short distance past the outer surface 208 of the stabilizing matrix 202. In FIG. 2b, it will be appreciated that the stabilizing matrix 202 may be hollowed out or otherwise removed directly above each detector 106 (and the light source 104) such that adequate reflected electromagnetic radiation is able to impinge upon each detector 106. This is shown in more detail below in FIG. 3c.

The stabilizing matrix 202 may be configured to be in direct contact with or substantially adjacent to the skin of the individual when the device 100 is in use. In operation, the stabilizing matrix 202 not only protects the light source 104 and detectors 106 from moisture and environmental contaminants, but it may also be configured to reduce vibration effects that would otherwise compromise the resulting PPG signal provided by the device 100. Motion artifacts can be particularly damaging to optoanalytical devices, such as the device 100 disclosed herein. The stabilizing matrix 202 may be configured to absorb all or a portion of these motion artifacts by reducing the motion of the detectors 106 with respect to the skin of the individual. To accomplish this, the stabilizing matrix 202 may be made of a pliant material. For example, in one embodiment, the stabilizing matrix 202 may be made of silicone or a type of silicone. In other embodiments, however, the stabilizing matrix may be made of other pliant materials such as, but not limited to, polymers (e.g., urethanes, etc.), elastomers (e.g., rubber, ethylene-vinyl acetate, etc.), soft plastics, foams, combinations thereof, or the like.

The stabilizing matrix 202 not only contributes to the reduction of motion artifacts, but may also serve to substantially seal off the detectors 106 from ambient light interference, which could likewise have a detrimental effect on the resulting PPG signal. To facilitate this, in some embodiments, the stabilizing matrix 202 may be made of a generally translucent or opaque material. It will be appreciated, however, that embodiments are nonetheless contemplated herein where the stabilizing matrix 202 is made of a generally transparent material. With a translucent or opaque material, however, external interferent light may be absorbed by the stabilizing matrix 202 instead of passing therethrough and thereafter impinging upon the detectors 106. To further seal off the detectors 106 from ambient light interference, one or more optical filters or films (not shown) may be applied to the outer surface 208 of the stabilizing matrix 202. In at least one embodiment, the optical filters or films may be arranged only over the detectors 106. As will be appreciated by those skilled in the art, optical filters and/or films may be useful in filtering out unwanted external light signals.

In operation, the device 100 may be used, for example, as a heart monitor, where the interaction of the light source 104 with the detectors 106 is configured to detect or otherwise record a heartbeat of an individual. To accomplish this, the device 100 may be arranged to measure the vasculature of an individual, and specifically the locally increasing and decreasing tissue blood volume that corresponds to the periodicity of heartbeats. Accordingly, the device 100 may be generally placed at locations on the body where a heartbeat is more apt to be detected. For example, the device 100 may be placed at the calf, the upper arm, the ankle, toes, fingers, the forehead, the chest, or any other suitable location on the body of the individual.

In at least one embodiment, the device 100 may be arranged on the wrist of the individual, either on the top or the underside of the wrist. To facilitate this, the device 100 may be used in conjunction with a commercially-available wristwatch or the like. For example, the device 100 may be coupled to the back-side of a wristwatch such that the device 100, and in particular the stabilizing matrix 202, is directed toward and in direct contact with the skin of the individual. The device 100 may be removably coupled to the watch using, for example, adhesives, mechanical fasteners, VELCRO®, magnetic attraction, suction devices, combinations thereof, or the like as applied to the back side 206 of the device 100. In at least one embodiment, a pressure-sensitive adhesive (not shown) or the like may be arranged on the outer surface 208 of the stabilizing matrix 202 in order to attain better contact/adhesion with the skin of the individual and also aid in the reduction of motion artifacts. Using a pressure sensitive adhesive may prove advantageous since it is typically long lasting, keeps adhesive properties in the presence of moisture and normal temperature variations, it is nonirritating, non-gumming, and non-peeling.

In operation, the light source 104 emits electromagnetic radiation into the skin of the individual to optically interact with the vasculature and thereby generate an optically interacted signal indicative of the amount of electromagnetic radiation absorbed by the hemoglobin in the blood. In at least one embodiment, the light source 104 is configured to emit red or infrared light. The device 100 relies on the differential absorbance of the electromagnetic radiation by different species of hemoglobin. The background absorbance of tissues and venous blood absorbs, scatters, and otherwise interferes with the absorbance directly attributable to the arterial blood. However, due to the enlargement of the cross-sectional area of the arterial vessels during the surge of blood from ventricular contraction, a relatively larger signal can be attributed to the absorbance of arterial hemoglobin during the systole. Whatever is not absorbed is either transmitted through the tissue, or reflected back to the detectors 106 for detection.

The processing device 110 (FIGS. 1a and 1b) may be communicably coupled to the detectors 106 and configured to receive the signal data generated by the detectors 106. In some embodiments, the processing device 110 may process the signal data and provide an output representative of cardiac-related information. In other embodiments, however, the processing device 110 may be configured to receive and wirelessly transmit the signal data to an adjacent computing system for processing. By averaging multiple readings derived from the detectors 106 and determining the ratio peaks of specific wavelengths, the relative absorbance due to the arterial blood flow may be estimated. First, by calculating the differences in absorption signals over short periods of time during which the systole and diastole are detected, the peak net absorbance by oxygenated hemoglobin is established. The software subtracts the major “noise” components (from non-arterial sources) from the peak signals to arrive at the relative contribution from the arterial pulse.

As appropriate, an algorithm may average readings, remove outliers, and/or increase or decrease the light intensity to obtain a result. In some embodiments, the algorithm may be configured to recognize when motion has interfered with the heartbeat signal, and in such cases the obstructed signal is then “zeroed,” thereby denoting that the data obtained was unanalyzable. Such calculations and determinations may be facilitated using, for example, the commercially-available signal measurement and analysis display software program LABVIEW™ or the like. The resulting calculations provide a measurement of arterial oxygen saturation in the vasculature of the individual, and also allows calculation of the shape of the pulse of the individual, which can be developed into a PPG trace. The PPG trace may then, in turn, be displayed on a screen associated with the device 100, as described below, or may be displayed for consideration by the adjacent machine-readable medium.

Referring now to FIGS. 3a and 3b, with continued reference to FIGS. 1a-b and 2a-c, illustrated is an exemplary housing 300 that may be used to house or otherwise retain the device 100, according to one or more embodiments. As illustrated in FIG. 3a, the housing 300 may include a body 302 having a front surface 303 and a back surface 305. The back surface 305 may define a recess 304 which extends to a bottom surface 306 thereof. The bottom surface 306 may define one or more ports 308 (three shown), which may substantially correspond to the ports 112 described above with reference to FIGS. 1a and 1b. Accordingly, the ports 308 may provide wired access to the device 100, such as via the one or more ports 112. The ports 308 may extend from the bottom surface 306 to a front surface 303 (substantially occluded in FIG. 3a) of the housing 300.

In some embodiments, the device 100 may be removably coupled to the back surface 305. In other embodiments, however, the recess 304 may be configured to receive or otherwise seat the device 100 therein, as shown in FIG. 3b. Accordingly, the back side 206 (FIG. 2a) of the device 100 may be configured to engage or otherwise substantially mate with the bottom surface 306 of the housing 300 when properly coupled. The device 100 may be removably coupled to the housing 300 using, for example, adhesives, mechanical fasteners, VELCRO®, combinations thereof, or the like. In at least one embodiment, however, as shown in FIG. 3c, the stabilizing matrix 202 may serve to hold the device 100 within the housing 300.

While the body 302 is shown as being generally circular in shape, it will be appreciated that any shape may be employed without departing from the scope of the disclosure. The body 302 may further include or otherwise define opposing elongate apertures 310a and 310b. The elongate apertures 310a,b may be configured to receive portions of a strap, belt, or band (not shown) used to attach the housing 300 to the wrist of an individual, similar to how a strap or band used on a wristwatch would function. The strap or band may function to hold the stabilizing matrix 202 (FIGS. 2a-c) into proper contact with the skin of the individual, and may be adjustable based on sizing. In one embodiment, the strap may be interweaved in the opposing elongate apertures 310a,b but also extend across the back side 305 of the housing 302, at least partially interposing the device 100 and the back side 305. Such an embodiment may help to apply an even pressure to the device 100 against the skin of the individual and thereby improve the resulting signal.

Referring to FIG. 3c, illustrated is an embodiment of the device 100 as arranged within the housing 300 and being substantially covered by the stabilizing matrix 202. As depicted, the stabilizing matrix 202 extends above the electrical components (not shown) of the device 100, and may serve at least partially to maintain the device 100 appropriately seated within the housing 300. One or more apertures 312 may be formed in the stabilizing matrix 202 above one or more of the light source 104 and/or the detectors 106 (not shown) in order to provide unobstructed emission and detection, respectively, of electromagnetic radiation. In some embodiments, the apertures 312 may define individualized “tunnels” within the stabilizing matrix 202 that allow the unobstructed emission and detection. In at least one embodiment, one or more of the tunnels may have a lens or other focusing device, or a filter arranged therein to improve signal detection.

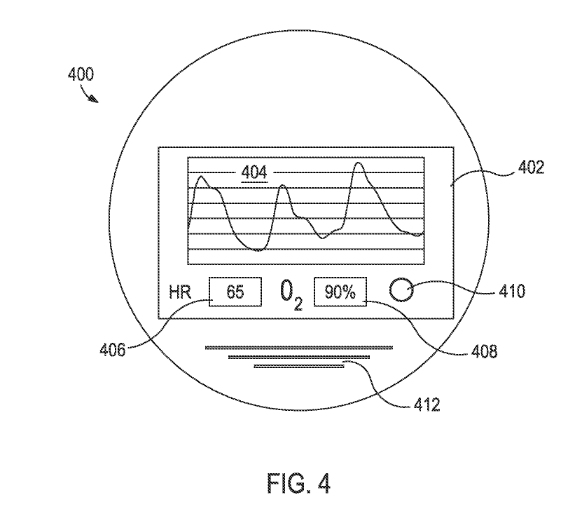

Referring now to FIG. 4, illustrated is an exemplary interface 400 that may be used in conjunction with the housing 300 described above, according to one or more embodiments. Specifically, the interface 400 may be coupled to or otherwise form part of the front surface 303 (FIG. 3a) of the housing and thereby be exposed to the user for visual reference while the device 100 is in use. As illustrated, the interface 400 may include an interactive display 402 configured to provide real-time cardiac-related information to the user. For example, in at least one embodiment, the display 402 may provide a real-time PPG trace 404, real-time heart rate 406 of the user, and real-time oxygen readings 408. Those skilled in the art will readily recognize, however, that the display 402 may be configured to provide any number of other cardiac-related informational components, without departing from the scope of the disclosure.

The interface 400 may also be configured with an indicator 410, such as an LED light. In some embodiments, the indicator 410 may be configured to flash different colors or varying flash patterns (e.g., predetermined patterns of flashes) to denote, for example, when the device 100 is in use or otherwise taking optical measurements or when the power supply 108 (FIGS. 1a-b) may need to be recharged. In other embodiments, the indicator 410 may be programmed with a color or flash pattern configured to inform the user if a signal is not detected, whether there is “zero” pulse, whether abnormal activity is detected, etc.

The interface 400 may also include an alarm 412 communicably coupled to the device 100 and configured to alert the user audibly or visually to one or more predetermined conditions measured by the device 100. In some embodiments, the alarm may take the form of a vibratory mechanism that could likewise alert the user to a measured predetermined condition. Generally, the alarm 412 may be configured to alert the user or others when any physiological parameter that is measured falls outside of a predetermined normal range such that appropriate action can be taken. For example, the alarm 412 may be configured to alert the user if the real-time heart rate or blood stream oxygen levels fall below a predetermined level or if the PPG trace indicates other cardiac-related problems. In such instances, the user can react by, for example, altering dietary intake or consuming prescribed medicines (e.g., for diabetes, high blood pressure, etc.). In some embodiments, the alarm 412 may be configured to alert a third party around the user that cardiac failure has occurred so that the third party can respond appropriately by, for example, calling 9-1-1, perform medical emergency procedures, etc.

In some embodiments, when the alarm 412 is triggered, the device 100 may be configured to alert or otherwise communicate with a hospital, a clinician, or an alert organization who would then take appropriate action to remedy the reported cardiac issue. This may be done wirelessly using, for example, BLUETOOTH® technology capable of communicating directly with a program on a computer, PDA, or smartphone device. The PDA or smartphone could then communicate with the hospital, clinician, or alert organization for continuous monitoring, analysis, or alarming of cardiac failure. In other embodiments, the device 100 may be self-recording and able to download to a computer or adjacent computing device capable of filtering and analyzing the signal data and subsequently communicating the alert to the hospital, clinician, or alert organization.

As can be appreciated, the device 100 can be readily adapted for both individual monitoring and clinical use. Individuals of all ages with a history of heart conditions can wear the device 100 during a majority of daily activities. Throughout the day their cardiac activity can be recorded discreetly and the resulting PPG trace and data can then either be monitored by the individual, or recorded and analyzed by a physician without the need for bulky vests or conspicuous devices. Additionally, the device 100 could readily be adapted to alert the appropriate medical personnel if cardiac failure or other predetermined cardiac conditions occur. As can be appreciated, this could be especially useful for the aging population, and those with serious heart conditions or a history of heart attacks. Furthermore, the device 100 may prove advantageous in a clinical setting for a comfortable and worry-free means of monitoring the cardiac activity of an individual for an extended period of time. In other applications, however, an individual without a noted heart condition could wear the device 100 for purposes of heart rate monitoring.

At least one aspect of the disclosure, the light source 104 and detectors 106 could be configured for near-infrared spectroscopy (“NIRS”). For example, in at least one embodiment, the light source 104 and detectors 106 could be configured to emit and detect, respectively, near-infrared wavelengths. NIRS can be useful in determining partial pressure, O₂ and CO₂ in the tissues, body pH, and other metabolic variables known by those skilled in the art.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

The invention claimed is:

1. A device, comprising:
a substrate;
at least one light source mounted on the substrate and configured to emit electromagnetic radiation that optically interacts with a vasculature and generates an optically interacted signal;
a plurality of detectors mounted on the substrate and configured to detect the optically interacted signal; and
a stabilizing matrix arranged on the substrate and substantially surrounding the at least one light source and the plurality of detectors, the stabilizing matrix being configured to absorb vibration and thereby reduce motion artifacts detectable by the plurality of detectors.

2. The device of claim 1, wherein the electromagnetic radiation is one of red or infrared light.

3. The device of claim 1, wherein the plurality of detectors form a circular array about the light source.

4. The device of claim 1, further comprising a processing device communicably coupled to the plurality of detectors and configured to receive signal data generated by the plurality of detectors.

5. The device of claim 5, wherein the processing device is a wireless transmitter configured to communicate the signal data to an adjacent computing system adapted to receive and process the signal data.

6. The device of claim 5, wherein the processing device is a microprocessor configured to receive and process the signal data.

7. The device of claim 1, wherein one or more of the plurality of detectors protrudes past an outer surface of the stabilizing matrix.

8. The device of claim 1, wherein the stabilizing matrix is made of a pliant material.

9. The device of claim 8, wherein the pliant material is silicone.

10. The device of claim 1, wherein the substrate is removably coupled to a back-side of a wrist watch such that the stabilizing matrix is in direct contact with skin of an individual.

11. A device, comprising:
a housing having a front surface and a back surface;
a substrate having a front side and a back side, the back side being removably coupled to the back surface of the housing;
at least one light source mounted on the front side of the substrate and configured to emit electromagnetic radiation that optically interacts with a vasculature and thereby generates an optically interacted signal;
a plurality of detectors mounted on the front side of the substrate and configured to detect the optically interacted signal; and
a stabilizing matrix arranged on the front side of the substrate and substantially surrounding the at least one light source and the plurality of detectors, the stabilizing matrix being configured to absorb vibration and thereby reduce motion artifacts detectable by the plurality of detectors.

12. The device of claim 11, wherein the back surface of the housing defines a recess configured to receive the substrate.

13. The device of claim 11, further comprising a layer of pressure-sensitive adhesive applied to an outer surface of the stabilizing matrix.

14. The device of claim 11, further comprising a processing device communicably coupled to the plurality of detectors and configured to receive signal data generated by the plurality of detectors.

15. The device of claim 14, further comprising an interface coupled to the front surface of the housing and communicably coupled to the processing device, the interface comprising an interactive display configured to provide real-time cardiac-related information to a user.

16. The device of claim 15, wherein the interface further comprises an alarm configured to alert the user to one or more predetermined cardiac conditions as detected by the plurality of detectors and measured by the processing device.

17. A method for detecting cardiac-related conditions, comprising:
emitting electromagnetic radiation through a vasculature using at least one light source mounted on a substrate, the electromagnetic radiation being configured to optically react with the vasculature and reflect an optically interacted signal;
detecting the optically interacted signal with a plurality of detectors mounted on the substrate, the plurality of detectors being configured to generate signal data;
absorbing vibration and reducing motion artifacts detectable by the plurality of detectors with a stabilizing matrix, the stabilizing matrix being arranged on the substrate and substantially surrounding the at least one light source and the plurality of detectors;
receiving the signal data with a processing device communicably coupled to the plurality of detectors; and
processing the signal data to determine the cardiac-related conditions.

18. The method of claim 17, wherein processing the signal data further comprises transmitting the signal data with the processing device to a computing device configured to analyze and filter the signal data and subsequently display a photoplethysmograph representative of the signal data.

19. The method of claim 17, wherein processing the signal data further comprises:
filtering and analyzing the signal data with the processing device to determine real-time cardiac-related information; and
displaying the real-time cardiac-related information on an interface communicably coupled to the processing device.

20. The method of claim 17, further comprising sealing off the plurality of detectors from ambient light interference using the stabilizing matrix.
